(19) United States
(12) Patent Application Publication
(10) Pub. No.: US 2011/0045103 A1
(43) Pub. Date: Feb. 24, 2011

(54) METHODS FOR TREATING AIDS

(76) Inventor: Abdul R. Abukurah, Northridge, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/859,708

(22) Filed: Aug. 19, 2010

Related U.S. Application Data
(60) Provisional application No. 61/235,290, filed on Aug. 19, 2009.

(51) Int. Cl. A61K 33/02; A61K 33/14; C12N 7/06

(52) U.S. Cl. 424/664; 435/238

(57) ABSTRACT

The present invention pertains to methods of inactivating human immunodeficiency virus (HIV), both in vitro and in vivo. Such methods also relate to treating HIV-related symptoms and the treatment of acquired immune deficiency syndrome in patients. Methods typically employ administering a composition comprising ammonium chloride such that HIV is inactivated.

### CROSS-REFERENCE TO RELATED APPLICATION

This application claims the benefit of priority of U.S. Provisional Application No. 61/235,290, filed Aug. 19, 2009, which is expressly incorporated herein by reference.

### BACKGROUND

The causative agent for AIDS (acquired immune deficiency syndrome) is known to be a virus of the retrovirus family called HIV (human immunodeficiency virus). Infection with HIV does not, however, immediately give rise to overt symptoms of AIDS. The only indication of exposure to the virus may be the presence of antibodies thereto in the blood of an infected subject. The infection may lie dormant, giving rise to no obvious symptoms, and the incubation period prior to development of AIDS may vary from several months to decades. A person infected with HIV gradually loses immune function along with certain immune cells called CD4 T-lymphocytes or CD4 T-cells, causing the infected person to become vulnerable to pneumonia, fungal infections, and other, common ailments. Development of AIDS itself may be preceded by the AIDS-related complex (ARC) which is characterized by unexplained fever, weight loss, chronic cough, or diarrhea.

The reasons for the variable period between infection with the virus and breakdown of the immune system in an infected individual are poorly understood. This progression can be monitored using surrogate markers (laboratory data that correspond to the various stages of disease progression) or clinical endpoints (illnesses associated with more advanced disease). Surrogate markers for the various stages of HIV infection include the declining number of CD4 T-cells. In general, the lower the infected person's CD4 T-cell count, the weaker the person's immune system and the more advanced the disease state. Factors presently unknown may trigger proliferation of the virus with consequential disruption of the immune system. The victims of the disease are then subject to various infections and malignancies that, unchecked by the disabled immune system, lead to death.

At this time, there is no cure for AIDS and management of the disease with FDA-approved drugs is extremely costly. As the scientific community continues to study HIV and search for ways to manage the symptoms of AIDS, it is hoped that less expensive medications can be identified and easily distributed not only to those in the United States but also to those in developing countries.

### SUMMARY

To address these and other needs in the art, the present invention generally provides methods of inactivating human immunodeficiency virus (HIV) in vitro or in vivo, and methods of treating HIV-positive patients and patients having acquired immune deficiency syndrome (AIDS).

Accordingly, in one general aspect, the present invention provides a method of inactivating at least 50% of HIV in an in vitro sample comprising contacting the sample with a composition comprising an effective amount of ammonium chloride.

Also contemplated are methods of treating patients. For example, the present invention provides a method of inactivating HIV in a patient comprising intravenously administering a composition comprising an effective amount of ammonium chloride to the patient.

In other embodiments, the present invention provides a method of treating a patient suffering from AIDS comprising intravenously administering a composition comprising an effective amount of ammonium chloride to the patient.

Another general embodiment of the present invention contemplates a method of delaying the onset of AIDS in an HIV-positive patient comprising intravenously administering a composition comprising an effective amount of ammonium chloride to the patient, wherein the onset of AIDS is delayed as compared to if the patient had not been intravenously administered the composition.

### DETAILED DESCRIPTION

Methods discussed herein offer an economical way to study HIV in vitro as well as to treat HIV-positive patients, such as patients having AIDS, using compositions comprising ammonium chloride. One aspect of the invention is directed to a method of inactivating at least 50% of HIV in an in vitro sample comprising contacting the sample with a composition comprising an effective amount of ammonium chloride. Another aspect of the invention is directed to a method of inactivating at least 50% of HIV in an in vitro sample comprising contacting the HIV with a composition comprising an effective amount of ammonium chloride. In certain embodiments, at least 75% of HIV is inactivated. In certain embodiments, at least 90% of HIV is inactivated. Other degrees of inactivation are discussed herein. HIV may be further defined as HIV-1 or HIV-2, or a combination thereof. In some embodiments, the sample contains HIV virions. In some embodiments, the sample comprises human T-cells, such as human T-cell leukemia cells.

A composition comprising ammonium chloride may have an amount of ammonium chloride ranging from 1-10 mg/ml, for example. In this or any other embodiment herein, a composition comprising ammonium chloride may be used for in vitro or in vivo methods. In certain embodiments, ammonium chloride is present in a composition in an amount of about, at most about, or at least about 1, 1.5, 2, 2.5, 3, 3.5, 4, 4.5, 5, 5.5, 6, 6.5, 7, 7.5, 8, 8.5, 9, 9.5, or 10 mg/ml, or any range derivable therein. In particular embodiments, a composition may comprise from 5 to 6 mg/ml, such as 5.36 mg/ml, ammonium chloride. Ammonium chloride may be present in a composition in amounts greater than 10 mg/ml, or less than 1 mg/ml. Other composition amounts are described herein.

A composition comprising ammonium chloride may contact the HIV in vitro or in vivo more than once in a defined time period. Dosing and dosage timing are described further herein.

Also contemplated is a method of inactivating HIV in a patient comprising intravenously administering a composition comprising an effective amount of ammonium chloride to the patient. In certain embodiments, the patient does not suffer from chronic kidney disease. In certain embodiments, the patient suffers from chronic kidney disease. Skilled artisans will understand that additional care should be taken when treating a patient suffering from a chronic kidney disease using methods described herein. This is because patients with chronic kidney disease suffer from metabolic acidosis, which can be aggravated by ammonium chloride administration. Hyperkalemia and the increased possibility of heart paralysis, making it stop in diastole, are risks associated with ammonium chloride administration to a chronic kidney disease patient. Thus, some methods contemplate administration of a composition comprising ammonium chloride that occurs for a time period that is less than the time period of administration if the patient did not suffer from chronic kidney disease. For example, administration may take place for 5, 10, 15, 20, or 30 minutes, or any range derivable therein, as opposed to, e.g., a 24-hour long administration of a composition of the same concentration to a patient that does not suffer from chronic kidney disease. In some embodiments, a composition comprising a reduced amount of ammonium chloride is administered, wherein the reduced amount is an amount that is reduced compared to the amount that would be administered if the patient did not suffer from chronic kidney disease, but is still effective to inactivate HIV as explained herein.

In any embodiment herein, a composition comprising ammonium chloride may further comprise an additional agent, such as sodium chloride. The concentration of sodium chloride in a composition may range from, e.g., about 0.5% to about 1.5%. In some embodiments, the sodium chloride concentration is about, at most about, or at least about 0.5%, 0.6%, 0.7%, 0.8%, 0.9%, 1.0%, 1.1%, 1.2%, 1.3%, 1.4%, or 1.5%. In some embodiments, the sodium chloride concentration is about 0.9%. Other optional agents are discussed herein.

Another aspect of the present invention contemplates a method of treating a patient suffering from AIDS comprising intravenously administering a composition comprising an effective amount of ammonium chloride to the patient.

The present invention also provides a method of delaying the onset of AIDS in an

HIV-positive patient comprising intravenously administering a composition comprising an effective amount of ammonium chloride to the patient, wherein the onset of AIDS is delayed as compared to if the patient had not been intravenously administered the composition. HIV-infected persons (that is, “HIV-positive persons”) are defined on clinical conditions associated with HIV infection and CD4+T lymphocyte counts. From a practical standpoint, the clinician views HIV infection as a spectrum of disorders ranging from primary infection with or without the acute HIV syndrome to the symptomatic infection state of advanced disease. The Centers for Disease Control and Prevention (CDC) in Atlanta, Ga., has established an authoritative definition for the diagnosis of AIDS, which informs a skilled artisan whether the onset of AIDS has occurred: in an HIV-positive individual, the CD4+T-cell count must be below 200 cells per cubic mm of blood, or there must be the clinical appearance of an initial AIDS-defining opportunistic infection, such as PCP (Pneumocystis Carinii Pneumonia), oral candidiasis (thrush), pulmonary tuberculosis, or invasive cervical carcinoma (cancer of the cervix in women). Methods of determining a patient's CD4+T-cell count are well-known in the art.

Persons of skill in the art are familiar with HIV inactivation. The Example below describes one method of assessing inactivation with respect to an in vitro sample. Another method of assessing HIV inactivation entails taking blood cultures followed by culturing in a T-Cell media and measuring the infectivity. An alternative method is to determine the virus copies that are present in the blood before and after the inactivation attempt or treatment in a periodic fashion (e.g., every 1-7 days). In a sample, about, at least about, or at most about 50%, 60%, 70%, 80%, 90%, 95%, 99%, or 100%, or any range derivable therein, of the virus may be inactivated. Methods of the invention also contemplate administering an effective amount of a composition comprising ammonium chloride, either in vitro to a sample or in vivo to a patient, until HIV is no longer detected.

The terms “contacted” and “exposed,” when applied to a cell or a sample, are used herein to describe the process by which a composition of the present invention is administered or delivered to a target cell or sample or is placed in direct juxtaposition with the target cell or sample. The terms “administered” and “delivered” are used interchangeably with “contacted” and “exposed.”

As used herein, the term “effective” (e.g., “an effective amount”) means adequate to accomplish a desired, expected, or intended result. For example, an “effective amount” may be an amount of a compound sufficient to produce a therapeutic benefit (e.g., effective to reproducibly inhibit decrease, reduce, or otherwise reduce the severity of an HIV infection). For any method comprising administration of an effective amount of a composition of the present invention to a patient, the method may alternatively comprise administration of a therapeutically effective amount of a composition, as that term is defined below.

As used herein, the term “patient” or “subject” refers to a living mammalian organism, such as a human, monkey, cow, sheep, goat, dogs, cat, mouse, rat, or guinea pig. In certain embodiments, the patient or subject is a primate. Non-limiting examples of human subjects are adults, juveniles, infants, and fetuses. A patient or subject may be further defined as one that suffers from chronic kidney disease. Alternatively, a patient or subject may be further defined as one that does not suffer from chronic kidney disease.

“Treatment” and “treating” as used herein refer to administration or application of a therapeutic agent, such as ammonium chloride, to a patient or performance of a procedure or modality on a patient for the purpose of obtaining a therapeutic benefit of a disease or health-related condition. For example, a patient infected with HIV may be subjected to a treatment comprising administration of a composition comprising ammonium chloride, as discussed herein, in order to mitigate HIV-related symptoms or to treat AIDS.

The term “therapeutic benefit” or “therapeutically effective amount” as used throughout this application refers to anything that promotes or enhances the well-being of the patient with respect to the medical treatment of a condition. This includes, but is not limited to, a reduction in the onset, frequency, duration, or severity of the signs or symptoms of a disease. For example, a therapeutically effective amount of a composition of the present invention may be an amount sufficient to provide a therapeutic benefit to an HIV-positive patient. Signs or symptoms of the initial stages of HIV infection include fever, swollen lymph nodes, sore throat, rash, muscle pain, malaise, and mouth and esophageal sores. AIDS, the final stage of HIV infection, may present with symptoms of various opportunistic infections, as is well-known in the art. Symptoms at this stage may include unexplained weight loss, recurring respiratory tract infections, prostatitis, skin rashes, and oral ulcerations. Methods of the claimed invention contemplate reducing the severity or duration of at least one HIV- or AIDS-related symptom comprising administering to an HIV-positive patient a composition comprising a therapeutically effective amount of ammonium chloride.

In any embodiment herein, the term “comprising” may be substituted with “consisting essentially of”or “consisting of.” For example, the present invention contemplates a method of inactivating at least 50% of HIV in a sample consisting essentially of contacting the sample with a composition comprising an effective amount of ammonium chloride. Also contemplated is a method of inactivating at least 50% of HIV in a sample consisting of contacting the sample with a composition comprising an effective amount of ammonium chloride. In such a method, it is intended that the method excludes other steps. Also contemplated is a method of inactivating at least 50% of HIV in a sample comprising contacting the sample with a composition consisting essentially of an effective amount of ammonium chloride. As another non-limiting example, also contemplated are methods of inactivating at least 50% of HIV in a sample consisting essentially of contacting the sample with a composition consisting of an effective amount of ammonium chloride. In such a method, it is intended that the composition excludes other ingredients. Another embodiment contemplates a method of inactivating at least 50% of HIV in a sample comprising contacting the sample with a composition consisting of an effective amount of ammonium chloride. In such a method, it is intended that the composition. excludes other ingredients. Another embodiment contemplates a method of inactivating at least 50% of HIV in a sample consisting of contacting the sample with a composition consisting of an effective amount of ammonium chloride. In such a method, it is intended that the method excludes other steps and that the composition excludes other ingredients. Also contemplated is a method of inactivating at least 50% of HIV in a sample consisting essentially of contacting the sample with a composition consisting essentially of an effective amount of ammonium chloride. Also contemplated is a method of inactivating at least 50% of HIV in a sample consisting of contacting the sample with a composition consisting essentially of an effective amount of ammonium chloride. In such a method, it is intended that the method excludes other steps. Other similar substitutions in any other embodiment discussed herein are also encompassed by the present invention. For those embodiments reciting “consisting essentially of,” it is noted that non-limiting examples of materials and steps that do not materially affect the basic and novel aspects of ammonium chloride include those that do not change the chemical structure of ammonium chloride, or those that do not decrease the effective amount of ammonium chloride that is administered either in vitro (e.g., that contacts a cell) or in vivo (e.g., that is intravenously administered). For those embodiments reciting “consisting of,” it is intended that other steps, components, or ingredients are excluded, as appropriate. It is further noted that any composition described herein may comprise, consist essentially of, or consist of sodium chloride or a pharmaceutically acceptable agent (e.g., carrier) in addition to an effective amount of ammonium chloride. Such carriers are described herein.

### Ammonium Chloride

Ammonium chloride, a relatively inexpensive agent compared to most FDA-approved AIDS drugs, may be obtained from a variety of commercial sources. For example, ammonium chloride injection, USP may be obtained at a concentration of 100 mEq (5 mEq/ml) from HOSPIRA, Inc., Lake Forest, Ill. Ammonium chloride has been approved by the FDA for use in treating patients that exhibit alkalosis. See, e.g., the world wide web at <dailymed.nlm.nih.govidailymed/drugInfo.cfm?id=1343>. Patients receiving ammonium chloride should be constantly observed for symptoms of ammonia toxicity (e.g., pallor, sweating, retching, irregular breathing, bradycardia, cardiac arrhythmias, local and general twitching, tonic convulsions, or coma). It should be used with caution in patients with high total CO₂ and buffer base secondary to primary respiratory acidosis. Intravenous administration should be slow, such as a rate that does not exceed 70 mL/hour to avoid local irritation and toxic effects. When exposed to low temperatures, concentrated solutions of ammonium chloride may crystallize. If crystals are observed, the vial should be warmed to room temperature in a water bath prior to use. Overdosage of ammonium chloride may result in a serious degree of metabolic acidosis, disorientation, confusion, or coma. As an example of a suitable dosage, one may administer about 100 mEq of ammonium chloride in one liter of a 0.9% sodium chloride solution for about 24 hours. Should metabolic acidosis occur following overdosage, the administration of an alkalinizing solution such as sodium bicarbonate or sodium lactate may serve to correct the acidosis.

### Pharmaceutical Formulations and Routes for Administration

Compositions comprising ammonium chloride that are administered to a patient infected with HIV are typically pharmaceutically or pharmacologically acceptable. A composition may comprise a pharmaceutically or pharmacologically acceptable agent in addition to ammonium chloride. The phrases “pharmaceutical or pharmacologically acceptable” refers to molecular entities and compositions that do not produce an adverse, allergic, or other untoward reaction when administered to a patient. The preparation of a pharmaceutical composition that contains ammonium chloride will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all solvents, dispersion media, surfactants, antioxidants, preservatives (e.g., antibacterial agents, antifungal agents), isotonic agents, salts, preservatives, drugs, drug stabilizers, gels, binders, excipients, disintegration agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions discussed herein is contemplated.

A composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

Sterile injectable solutions may be prepared by incorporating the active compound (e.g., ammonium chloride) in the required amount in the appropriate solvent (e.g., water) with various of the other ingredients described herein, as required, followed by filtered sterilization. Generally, dispersions are prepared by incorporating the various sterilized active ingredients into a sterile vehicle which contains the basic dispersion medium or the other ingredients. In the case of sterile powders for the preparation of sterile injectable solutions, suspensions, or emulsions, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent (e.g., water) first rendered isotonic prior to injection with sufficient saline or glucose.

The composition is preferably stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

The actual dosage amount of a composition of the present invention administered to a patient can be determined by physical and physiological factors such as body weight, severity of condition, previous or concurrent therapeutic interventions, idiopathy of the patient, and on the route of administration. The practitioner responsible for administration will, in any event, determine the concentration of active ingredient(s) in a composition and appropriate dose(s) for the individual patient. For example, dosing may be determined by following laboratory studies of blood gases to determine acidity and electrolyte status, or the composition of sodium, potassium, chloride, or CO₂, as well as lymphocyte count and CD4-T cell counts over the treatment period to see the effective suppression or inactivation of the virus by the ammonium chloride composition.

When administering a composition comprising an effective amount or a therapeutically effective amount of ammonium chloride to a patient, administration is typically intravenously. The intravenous administration of ammonium chloride may, in certain embodiments, be 100 mEq of ammonium chloride vial (e.g., 20 ml) added to 1 liter of 0.9% saline solution. While dosing is discussed further below, ammonium chloride may be administered intravenously over a period of 24 hours, for example.

The dose can be repeated as needed as determined by those of ordinary skill in the art. Thus, in some embodiments set forth herein, a single dose is contemplated. In other embodiments, two or more doses are contemplated. Where more than one dose is administered to a patient, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges. In certain embodiments, it may be desirable to provide a continuous supply of a pharmaceutical composition to the patient.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. As suggested above, within a single day (24-hour period), the patient may be given one or multiple administrations of the agent(s). Moreover, after a course of treatment, it is contemplated that there may be a period of time at which no other treatment is administered. This time period may last 1, 2, 3, 4, 5, 6, 7 days, or 1, 2, 3, 4, 5 weeks, or 1, 2, 3, 4, 5, 6, 7, 8, 9, 10, 11, 12 months or more, depending on the condition of the patient, such as their prognosis, strength, health, etc.

In particular embodiments, a composition may be administered 1, 2, 3, 4, 5, 6, 7, 8, 9, 10, 11, 12, 13, 14, 15, 16, 17, 18, 19, or 20 or more times, or every 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, or 7 days, or 1, 2, 3, 4, or 5 weeks, or 1, 2, 3, 4, 5, 6, 7, 8, 9, 10, 11, or 12 months or more, or any range or combination derivable therein.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

As used herein, “a” or “an” means one or more, unless clearly indicated otherwise.

Throughout this application, the term “about” is used to indicate that a value includes the standard deviation of error for the device and/or method being employed to determine the value.

### EXAMPLE 1

Antiviral Study of Ammonium Chloride Against HIV Type 1

Composition Tested: A 20 mL vial of ammonium chloride (100 mEq/5 mL) was dissolved into a 1 liter bag of 0.9% sodium chloride injection USP (9 g/L sodium chloride; osmolarity of 308 mOsmol/L; 154 mEq/L sodium; 154 mEq/L chloride). The contents of the bag were transferred to a sterile glass bottle. The solution was equilibrated to room temperature prior to use.

Virus: human immunodeficiency virus (HIV) Type I, strain HTLV-III_{B}, was obtained from Advanced Biotechnologies, Inc., Columbia, Md. Stock virus was prepared by collecting the supernatant culture fluid from infected culture cells. The cells were disrupted and cell debris removed by centrifugation at approximately 2200 RPM for ten minutes at room temperature. The supernatant was removed, aliquoted, and the high titer stock virus was stored at ≦−70° C. until the day of use. On the day of use, an aliquot of stock virus was removed, thawed, and maintained at a refrigerated temperature until used in the assay. The stock virus culture was adjusted to contain 5% fetal bovine serum as the organic soil lead. The stock virus tested demonstrated cytopathic effects (CPE) typical of HIV on MT-2 cells.

Test Cultures: MT-2 cells (human T-cell leukemia cells) were obtained through the AIDS Research and Reference Reagent Program, Division of AIDS, NIAID, NIH from Dr. Douglas Richman. Cultures were maintained and used in suspension in tissue culture labware at 36-38° C. in a humidified atmosphere of 5-7% CO₂.

Test Medium: The test medium used for the assay was RPMI 1640 supplemented with 10% (v/v) heat-inactivated fetal bovine serum (FBS). The medium was also supplemented with 2.0 mM L-glutamine and 50 μg/mL gentamicin.

TABLE 1
Parameters tested for virucidal efficacy assay.

| Test or Control | Dilutions Assayed | Cultures per | Total |
|---|---|---|---|
| Group | (log₁₀) | Dilution | Cultures |
| Cell Control | N/A | 4 | 4/group |
| Virus Control | −2, −3, −4, −5, −6, −7 | 4 | 24 |
| Sample lot + virus | −2, −3, −4, −5, −6, −7 | 4 | 24 |
| (Test composition) | | | |
| Cytotoxicity | −2, −3, −4 | 4 | 12 |
| Control | | | |
| Neutralization | −2, −3, −4 | 4 | 12 |
| Control | | | |

Treatment of Virus Suspension: A 1.80 mL aliquot of the test substance was dispensed into a sterile tube and mixed with a 0.2 mL aliquot of the stock virus suspension. The mixture was vortex mixed for 10 seconds and held for the remainder of the specified exposure time at 37.0° C. The exposure time was ten minutes. Following the exposure time, a 0.1 mL aliquot was removed from the tube and the mixture was immediately titered by 10-fold serial dilution (0.1 mL+0.9 mL test medium) and assayed for the presence of virus in MT-2 cells. To decrease the test substance cytotoxicity, the first dilution was made in FBS with the remaining dilutions in test medium.

Treatment of Virus Control: A 0.2 mL aliquot of stock virus suspension was exposed to a 1.80 mL aliquot of test medium, in lieu of test substance, and treated as previously described. Following the exposure time, a 0.1 mL aliquot was removed from the tube and the mixture was immediately titered by 10-fold serial dilution (0.1 mL+0.9 mL test medium) and assayed for the presence of virus in MT-2 cells. All controls employed the FBS neutralizer as described in the Treatment of Virus Suspension section above. The virus control titer was used as a baseline to compare the percent and log reductions of the test parameter following exposure to the test substance.

Cytotoxicity Control: A 1.80 mL aliquot of the test substance was mixed with a 0.2 mL aliquot of test medium containing the BSA organic soil load, in lieu of virus, and treated as previously described. The cytotoxicity control was held for the ten minute exposure time. Following the exposure time, a 0.2 mL aliquot was removed from the tube and the mixture was immediately titered by 10-fold serial dilution (0.2 mL+1.8 mL test medium) in MT-2 cells. The cytotoxicity of the MT-2 cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Control: Each cytotoxicity control mixture (above) was challenged with low titer stock virus to determine the dilution(s) of test substance at which virucidal activity, if any, was retained. Dilutions that showed virucidal activity were not considered in determining reduction of the virus by the test substance. Using the cytotoxicity control dilutions prepared above, an additional set of indicator cell cultures was inoculated with a 0.2 mL aliquot of each dilution in quadruplicate. A 0.02 mL aliquot of low titer stock virus was inoculated into each cell culture well and the indicator cell cultures were incubated along with the test and virus control plates.

Infectivity Assay: The MT-2 cell line, which exhibits CPE in the presence of HIV-1, was used as the indicator cell line in the infectivity assays. Cells in the multiwall culture dishes were inoculated in quadruplicate with 0.2 mL of the dilutions prepared from test and control groups. Uninfected indicator cell cultures (cell controls) were inoculated with test medium alone. The cultures were incubated at 36-38° C. in a humidified atmosphere of 5-7% CO₂ in sterile disposable cell culture labware. The cultures were scored periodically for eight days for the absence or presence of CPE, cytotoxicity, and for viability.

Data analysis: Viral and cytotoxicity titers are expressed as −log ₁₀ of the 50 percent titration endpoint for infectivity (TCID₅₀) or cytotoxicity (TCD₅₀), respectively, as calculated by the method of Spearman Karber:

$$Log of 1 st dilution inoculated - [ ( ( \frac{\begin{matrix}Sum of \% mortality \\ at each dilution\end{matrix}}{100} ) - 0.5 ) \times ( logarithm of dilution ) ]$$

### Calculation of Percent (%) Reduction:

$$\% Reduction = 1 - [ \frac{{TCID}_{50} test}{{TCID}_{50} virus control} ] \times 100$$

### Calculation of Log Reduction:

TCID₅₀ of the virus control—TCID₅₀ of the test substance=Log reduction Study Acceptance Criteria: A valid test required the following: (1) that stock virus be recovered from the virus control; (2) that the cell controls be negative for virus; and (3) that negative cultures are viable.

Results: Test substance cytotoxicity was not observed at any dilution tested (≦1.5 log ₁₀). The neutralization control demonstrated that the test substance was neutralized at ≦1.5 log ₁₀. The titer of the virus control was 6.0 log ₁₀. Following exposure, test virus infectivity was detected in the virus-test substance mixture at 5.0 log ₁₀.

Using a composition of 100 mEq/5 mL ammonium chloride (20 mL) dissolved in 1 liter of 0.9% Sodium Chloride Injection USP in the presence of 5% fetal bovine serum, a 90% reduction in viral titer was observed following a ten minute exposure time at 37° C. of the composition to HIV Type-1 as compared to the titer of the virus control. The log reduction in viral titer was 1.0 log ₁₀.

TABLE 2
Assay results.

| | | Test: HIV Type | | |
|---|---|---|---|---|
| | | 1 + Ammonium | | |
| | Virus | Chloride | Cytotoxicity | Neutralization |
| Dilution | Control | (100 mEq/5 mL) | Control | Control |
| Cell Control | 0 0 0 0 | 0 0 0 0 | 0 0 0 0 | 0 0 0 0 |
| 10⁻² | + + + + | + + + + | 0 0 0 0 | + + + + |
| 10⁻³ | + + + + | + + + + | 0 0 0 0 | + + + + |
| 10⁻⁴ | + + + + | + + + + | 0 0 0 0 | + + + + |
| 10⁻⁵ | + + + + | 0 0 + + | NT | NT |
| 10⁻⁶ | + + 0 0 | 0 0 0 0 | NT | NT |
| 10⁻⁷ | 0 0 0 0 | 0 0 0 0 | NT | NT |
| TCID₅₀/ | 10^{6.0} | 10^{5.0} | ≦10¹⁵* | Neutralized at |
| 0.2 mL | | | | a TCID₅₀ |
| | | | | of ≦1.5 Log₁₀ |
| Percent | NA | 90.0% | NA | NA |
| Reduction | | | | |
| Log₁₀ | NA | 1.0 Log₁₀ | NA | NA |
| Reduction | | | | |

+ = Positive for the presence of test virus
0 = No test virus recovered and/or no cytotoxicity present
NA = Not applicable
NT = Not tested
*= Cytotoxicity results are reported as TCD₅₀/0.2 mL

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of inactivating at least 50% of human immunodeficiency virus (HIV) in an in vitro sample comprising contacting the sample with a composition comprising an effective amount of ammonium chloride.

2. The method of claim 1, wherein at least 75% of HW is inactivated.

3. The method of claim 1, wherein at least 90% of HW is inactivated.

4. The method of claim 1, wherein the HIV is further defined as HIV-1 or HIV-2, or a combination thereof.

5. The method of claim 1, wherein the sample comprises human T-cells.

6. The method of claim 1, wherein ammonium chloride is present in the composition in an amount ranging from 1-10 mg/ml.

7. The method of claim 1, wherein the composition contacts the HIV more than once in a defined time period.

8. A method of inactivating human immunodeficiency virus (HIV) in a patient comprising intravenously administering a composition comprising an effective amount of ammonium chloride to the patient.

9. The method of claim 8, wherein the patient does not suffer from chronic kidney disease.

10. The method of claim 8, wherein the patient suffers from chronic kidney disease.

11. The method of claim 10, wherein administration occurs for a time period that is less than the time period of administration if the patient did not suffer from chronic kidney disease.

12. The method of claim 8, wherein the composition further comprises sodium chloride.

13. A method of treating a patient suffering from acquired immune deficiency syndrome (AIDS) comprising intravenously administering a composition comprising an effective amount of ammonium chloride to the patient.

14. A method of delaying the onset of acquired immune deficiency syndrome (AIDS) in a human immunodeficiency virus (HIV)-positive patient comprising intravenously administering a composition comprising an effective amount of ammonium chloride to the patient, wherein the onset of AIDS is delayed as compared to if the patient had not been intravenously administered the composition.
